(19) United States
(12) Patent Application Publication
(10) Pub. No.: US 2012/0184705 A1
(43) Pub. Date: Jul. 19, 2012

(54) METHOD FOR PRODUCING POLYOLS CONTAINING AMINO GROUPS

(75) Inventors: Klaus Lorenz, Dormagen (DE); Manfred Schmidt, Dormagen (DE)

(73) Assignee: Bayer MaterialScience AG, Leverkusen (DE)

(21) Appl. No.: 13/498,772

(22) PCT Filed: Sep. 27, 2010
(86) PCT No.: PCT/EP2010/064275

§ 371 Date: Mar. 28, 2012

(30) Foreign Application Priority Data
Sep. 29, 2009 (DE) 10 2009 043 616.2

(51) Int. Cl. C08G 18/32; C07C 217/08; C07C 213/04

(52) U.S. Cl. 528/85; 564/475; 564/505

(57) ABSTRACT

The present invention provides a simple method with no processing for producing polyols based on amino group-containing starter compounds. Unless explicitly specified, polyols are understood to be polyether polyols, polyether ester polyols and also polyether ester amide polyols. The invention also provides the polyols obtainable by the method according to the invention and the use of the polyols according to the invention to produce polyurethane materials.

The present invention provides amino group-containing polyols that are obtainable by a simple method. Unless explicitly specified, polyols within the meaning of the invention are understood to be polyether polyols, polyether ester polyols and also polyether ester amide polyols. The invention also provides the method for producing the amino group-containing polyols themselves as well as the use of the amino group-containing polyols according to the invention to produce polyurethane materials.

Suitable polyols for the production of polyurethane materials such as flexible or rigid foams or solid materials such as elastomers are generally obtained by polymerisation of suitable alkylene oxides onto polyfunctional starter compounds (i.e. containing a plurality of Zerewitinoff-active hydrogen atoms). Very diverse methods have long been known for performing these polymerisation reactions, some of which complement one another:

The base-catalysed addition of alkylene oxides to starter compounds containing Zerewitinoff-active hydrogen atoms is of importance in industry, whilst the use of double metal cyanide compounds (DMC catalysts) to perform this reaction is also gaining in importance. The use of highly active DMC catalysts, which are described for example in U.S. Pat. No. 5,470,813, EP-A 700 949, EP-A 743 093, EP-A 761 708, WO-A 97/40086, WO-A 98/16310 and WO-A 00/47649, makes polyether polyol production possible at very low catalyst concentrations (25 ppm or less), so that separation of the catalyst from the finished product is no longer necessary. However, these catalysts are not suitable for the production of short-chain polyols or of polyols based on starters containing amino groups. Basic catalysts, based for example on alkali metal hydroxides, have long been known and allow the straightforward production of short-chain polyols and/or polyols based on amino group-containing starters; however, the catalyst, i.e. the polymerisation-active sites on the polyether chains, has/have to be deactivated by neutralisation, for example. If a salt that is insoluble in the polyether polyol is formed in this process, it generally has to be isolated by means of a separate processing step, for example by filtration.

In the production of amino group-containing polyols in particular, products with a yellow to yellow-brown coloration are often obtained in this process; coloured starting materials are not desirable for certain applications, e.g. in paints and coatings. The polymerisation of alkylene oxides catalysed by (Lewis) acids is of lesser importance for starter compounds containing amino groups.

The base-catalysed addition of alkylene oxides such as for example ethylene oxide or propylene oxide to starter compounds containing Zerewitinoff-active hydrogen atoms takes place, as already mentioned, in the presence of alkali metal hydroxides, but alkali metal hydrides, alkali metal carboxylates, alkaline-earth hydroxides or amines such as for example N,N-dimethylbenzylamine or imidazole or imidazole derivatives can also be used. In the case of amino group-containing starters having Zerewitinoff-active hydrogen atoms bonded to nitrogen atoms, up to one mol of propylene oxide per mol of Zerewitinoff-active hydrogen atoms can be added without catalysis; if this ratio is exceeded then one of the aforementioned basic catalysts generally has to be added. Following addition of the alkylene oxides the polymerisation-active sites on the polyether chains have to be deactivated. There are various possible procedures for this. For example, they can be neutralised with dilute mineral acids such as sulfuric acid or phosphoric acid. The strength of the second dissociation step of sulfuric acid is sufficient to neutralise the alkali metal hydroxides produced by hydrolysis of the active alcoholate groups, such that 2 mol of alcoholate groups can be neutralised per mol of sulfuric acid used. Phosphoric acid, by contrast, has to be used in the equimolar amount to the amount of alcoholate groups to be neutralised. The salts that are formed during neutralisation and/or during removal of the water by distillation generally have to be isolated by means of filtration processes. Distillation and filtration processes are time-consuming and energy-intensive, and moreover in many cases they are not readily reproducible. For that reason many methods have been developed which manage without a filtration step and in many cases also without a distillation step: neutralisation with hydroxycarboxylic acids such as lactic acid for example is described in WO-A 98/20061 and US-A 2004167316 for the processing of short-chain polyols for rigid foam applications; these are widely used and well-established methods. U.S. Pat. No. 4,521,548 describes how the polymerisation-active sites can be deactivated in a similar manner by reacting with formic acid. The metal carboxylates that form after neutralisation with hydroxycarboxylic acids or formic acid dissolve to a clear solution in the polyether polyols. The disadvantage of these methods, however, is the catalytic activity of the salts remaining in the products, which is undesirable for many polyurethane applications. In WO-A 04/076529 the polymerisation reactions are therefore performed at low catalyst concentrations of 10 to 1000 ppm KOH, so that catalytically active hydroxycarboxylic salts remaining in the polyol after neutralisation are likewise present in low concentrations and so are less disruptive in subsequent reactions. In JP-A 10-30023 and U.S. Pat. No. 4,110,268 aromatic sulfonic acids or organic sulfonic acids are used for neutralisation which likewise form soluble salts in the polyether polyols but which are less basic and are distinguished by low catalytic activity. The high costs of the sulfonic acids represent a critical disadvantage here. The method for neutralising polyether polyols with sulfuric acid that is used in EP-A 2028211 proceeds in such a way that relatively large amounts of acidic sulfuric acid salts are obtained. The disadvantage here is that in the case of polyether polyols based on starter compounds containing amino groups, the products obtained are too turbid for many applications. The method described in WO-A 2009/106244 for neutralising polyethers with monobasic acids, which does not include a filtration step, requires the presence of at least 10 wt. % of ethylene oxide units (oxyethylene units) in the polyether chains, otherwise turbid products are obtained in this case too. Processing with acid cation exchangers as described in DE-A 100 24 313 requires the use of solvents and their removal by distillation, and is thus likewise associated with high costs. Phase separation methods require a hydrolysis step but no neutralisation step and are described for example in WO 01/14456, JP-A 6-157743, WO 96/20972 and U.S. Pat. No. 3,823,145. The phase separation of the polyether polyols from the alkaline aqueous phase is supported by the use of coalescers or centrifuges, but solvents often have to be added in this case too in order to increase the density difference between the polyether phase and the aqueous phase. Such methods are not suitable for all polyether polyols; in particular they do not work with short-chain polyether polyols or polyether polyols having high proportions of ethylene oxide. The use of solvents is expensive, and centrifuges have a high maintenance requirement.

In the case of amine-catalysed alkylene oxide addition reactions no further processing is necessary, provided that the presence of amines in these polyols does not adversely impair the production of polyurethane materials. Only polyols with relatively low equivalent weights can be obtained by amine catalysis, see for example in this regard Ionescu et al. in “Advances in Urethane Science & Technology”, 1998, 14, p. 151-218.

The object of the present invention was therefore to develop a production method for amino group-containing polyols produced with alkali or alkaline-earth hydroxide, carboxylate or hydride catalysis, which is characterised by an inexpensive processing method and which does not have the disadvantages of the prior art methods, elevated turbidity values and poor suitability for producing isocyanate prepolymers. The aim of the invention was therefore a production method for amino group-containing polyols having low turbidity values which can additionally be processed to give isocyanate group-containing prepolymers having improved storage stability.

Surprisingly this object could be achieved by a method which is characterised in that
(i) alkylene oxide mixtures containing alkylene oxides other than ethylene oxide or a maximum of 9 wt. % of ethylene oxide are added to amino group-containing starter compounds in the presence of a catalyst selected from at least one of the group consisting of alkali metal hydroxide, alkali metal hydride, alkaline-earth metal hydride, alkali metal carboxylate, alkaline-earth metal carboxylate and alkaline-earth hydroxide,
(ii) then the basic catalyst residues are neutralised by adding the stoichiometric amount of one or more monobasic inorganic acids and
(iii) the salts formed are left in the resulting polyol.

This procedure produces clear products which surprisingly have a lower tendency to develop turbidity than corresponding polyols neutralised with polybasic acids. Furthermore, isocyanate prepolymers obtained on the basis of polyols neutralised with monobasic inorganic acids by reaction with hyperstoichiometric amounts of isocyanates surprisingly exhibit better storage stability than corresponding isocyanate prepolymers based on polyols neutralised with polybasic acids. The method can be used with long- and short-chain polyols, in other words the OH value range of the end products extends from approximately 20 mg KOH/g to approximately 1000 mg KOH/g. The structure of the polyether chains, i.e. the composition of the alkylene oxides or alkylene oxide mixture used to produce the polyols, can likewise be varied within the framework of the aforementioned upper limit of maximum 9 wt. % of oxyethylene groups in the polyether chains, i.e. the oxyethylene groups can be arranged in blocks or randomly between the other alkylene oxide units.

The method according to the invention is performed in detail as follows:

The starter compounds are conventionally placed in the reactor, and the catalyst, i.e. the alkali metal hydroxide, alkali or alkaline-earth metal hydride, alkali or alkaline-earth metal carboxylate or alkaline-earth hydroxide, is optionally added at this point. Alkali metal hydroxides are preferably used, potassium hydroxide being particularly preferred. The catalyst can be introduced into the starter compound(s) as an aqueous solution or as a solid. The catalyst concentration relative to the amount of end product is preferably 0.004 to 0.11 wt. %, particularly preferably 0.01 to 0.11 wt. %, most particularly preferably 0.025 to 0.11 wt. %. The solution water and/or the water released during the reaction of the starter compounds with the catalyst can be removed under vacuum at elevated temperature, preferably at the reaction temperature, before the start of metering of the alkylene oxide(s), provided that the starter compounds used have a sufficiently low vapour pressure. Alternatively, alkylene oxide can initially be added without catalyst to the amino group-containing starter compounds and the alkali metal hydroxide added and the water removal step performed only once the starter species reach a sufficiently low vapour pressure. With low catalyst concentrations the water removal step can also be omitted.

Pre-prepared alkylene oxide addition products of starter compounds containing Zerewitinoff-active hydrogen atoms and having alkoxylate contents of 0.05 to 50 eq. % (“polymeric alkoxylates”) can also be used as basic catalysts. The alkoxylate content of the catalyst is understood to be the proportion of Zerewitinoff-active hydrogen atoms removed by a base by deprotonation relative to all Zerewitinoff-active hydrogen atoms originally present in the alkylene oxide addition product of the catalyst. The amount of polymeric alkoxylate used is naturally dependent on the catalyst concentration required for the amount of end product, as described in the previous paragraph.

Hydrogen bonded to N, O or S is referred to as Zerewitinoff-active hydrogen (sometimes also simply as “active hydrogen”) if it yields methane when reacted with methyl magnesium iodide by a method discovered by Zerewitinoff. Typical examples of compounds containing Zerewitinoff-active hydrogen are compounds containing carboxyl, hydroxyl, amino, imino or thiol groups as functional groups.

The polymeric alkoxylates suitable for use as the catalyst are produced in a separate reaction step by alkylene oxide addition to starter compounds containing Zerewitinoff-active hydrogen atoms. An alkali or alkaline-earth metal hydroxide, e.g. KOH, in amounts from 0.1 to 1 wt. %, relative to the amount of catalyst to be produced, is conventionally used in the production of the polymeric alkoxylate; the reaction mixture is dewatered under vacuum if necessary, the alkylene oxide addition reaction is performed under an inert gas atmosphere at 100 to 170° C. until an OH value of 150 to 1200 mg KOH/g is achieved, and then the polymeric alkoxylate is optionally adjusted to the aforementioned alkoxylate contents of 0.05 to 50 eq. % by adding further alkali or alkaline-earth metal hydroxides and then removing the water. Polymeric alkoxylates produced in such a way can be stored separately under an inert gas atmosphere. They are particularly preferably used in the method according to the invention if substances are used which are susceptible to hydrolysis under alkaline conditions or if the amount of low-molecular-weight starter in the production of long-chain polyols is not sufficient to guarantee adequate mixing or cooling of the reaction mixture at the start of the reaction. The amount of polymeric alkoxylate used in the method according to the invention is conventionally determined such that it corresponds to an alkali or alkaline-earth metal hydroxide concentration of preferably 0.004 to 0.11 wt. %, particularly preferably 0.01 to 0.11 wt. %, most particularly preferably 0.025 to 0.11 wt. %, relative to the amount of end product according to the invention to be produced. The polymeric alkoxylates can of course also be used as mixtures. The polymeric alkoxylates suitable for catalysing the alkylene oxide addition to amino group-containing starter compounds can also be obtained from amino group-free starter compounds.

The starter compounds placed in the reactor are then reacted with alkylene oxides under an inert gas atmosphere at temperatures of 80 to 180° C., preferably 100 to 170° C. The reaction temperature can of course be varied within the specified limits during the alkylene oxide metering phase: for example, in order to obtain an optimum balance between high epoxide conversion and low by-product formation, the alkylene oxide(s) can be added at high temperatures in the range of relatively low molar masses, at lower temperatures in the range of high molar masses, and post-reactions performed in turn at higher temperatures. The temperature of the exothermic alkylene oxide addition reaction is held at the desired level by cooling. According to the prior art relating to the design of polymerisation reactors for exothermic reactions (e.g. Ullmann's Encyclopedia of Industrial Chemistry, vol. B4, page 167ff, 5th edition, 1992), this type of cooling generally takes place via the reactor wall (e.g. double-walled jacket, half-pipe coil jacket) and by means of further heat-exchange surfaces positioned internally in the reactor and/or externally in the pump circuit, for example on cooling coils, bayonet coolers, plate-type, shell-and-tube or mixer heat exchangers. These should be designed in such a way that effective cooling is possible at the start of the metering phase too, i.e. with low fill levels.

As a general rule it is important to ensure thorough mixing of the reactor contents in all phases of the reaction through the design and use of commercial stirring devices, wherein in particular single-stage or multi-stage stirrers or stirrer types acting over a large area of the fill height are suitable (see for example Handbuch Apparate; Vulkan-Verlag Essen, 1st edition (1990), p. 188-208). Of particular importance in industry is a volume-specific mixing power input averaged across the entire reactor contents generally in the range from 0.2 to 5 W/l, with correspondingly higher volume-specific local power inputs in the vicinity of the stirring devices themselves and optionally with lower fill levels. According to the general prior art, a combination of baffles (e.g. flat or tubular baffles) and cooling coils (or bayonet coolers) can be arranged in the reactor to optimise the stirring action; these can also extend beyond the base of the vessel. The stirring power of the mixing unit can also be varied during the metering phase according to the fill level and degree of alkoxylation in order to ensure a particularly high energy input in critical reaction phases. Alkylene oxide addition products based on amino group-containing starter compounds often pass through a viscosity maximum for example once approximately 1 mol of alkylene oxide has been added per mol of NH hydrogens. Particularly intensive mixing is of course advantageous at that point. Stirring stages flush to the base and stirring devices flush to the wall are preferably used here. In addition, the stirrer geometry should help to reduce foaming of reaction products, for example after the metering and secondary reaction phase on separation of residual epoxides under vacuum. Stirring devices which achieve a continuous mixing of the surface of the liquid have proved suitable here. Depending on requirements, the stirrer shaft has a floor bearing and optionally further thrust bearings in the vessel. The stirrer shaft can be driven from above or below (with the shaft positioned centrically or eccentrically).

Alternatively, it is naturally also possible to achieve the necessary mixing exclusively by means of a pump circuit passing through a heat exchanger, or to operate this system as an additional mixing component in addition to the stirring unit, wherein the reactor contents are circulated according to requirements (typically 1 to 50 times per hour).

Many different types of reactor are suitable in general for performing the method according to the invention. Cylindrical vessels having a height to diameter ratio of 1:1 to 10:1 are generally used. Spherical, dished, flat or conical bases for example are suitable as reactor bases.

The alkylene oxides are fed continuously into the reactor in the customary way such that the safe pressure limits of the reactor system used are not exceeded. These are naturally dependent on the apparatus used in individual cases; the process is preferably generally performed in a pressure range between 1 mbar and 10 mbar, the pressure range from 1 mbar to 4 mbar being particularly preferred. The alkylene oxide(s) can be introduced into the reactor in various ways: metering into the gas phase or directly into the liquid phase is possible, for example via a submerged pipe or a diffuser ring located in the vicinity of the reactor base in a well-mixed zone. If a mixture of alkylene oxides is added, the individual alkylene oxides can be introduced into the reactor separately or as a mixture. Premixing of the alkylene oxides can be carried out for example using a mixing unit located in the common metering section (inline blending). It has also proved effective to meter alkylene oxides individually or in premixed form into the pump circuit on the pump pressure side. To ensure thorough mixing with the reaction medium it is then advantageous to include a high-shear mixing unit in the alkylene oxide/reaction medium flow.

If, as was mentioned above, a certain proportion of the alkylene oxide(s) is to be added without catalyst at the start, metering of the alkylene oxide(s) must be interrupted at the appropriate point and the catalyst added at the end of an appropriate post-reaction time. The optionally second alkylene oxide metering phase is followed by a (second) post-reaction phase in which the remaining alkylene oxide reacts. The end of this post-reaction phase is reached when no further pressure drop can be detected in the reaction vessel. Residual epoxide contents can optionally then also be removed by means of a vacuum, inert gas or steam stripping step.

The alkaline alkylene oxide addition product can then initially be hydrolysed with water. However, this hydrolysis step is not essential to the performance of the method according to the invention. The amount of water is up to 15 wt. %, relative to the amount of alkaline alkylene oxide addition product. Neutralisation of the alkaline polymerisation-active sites of the crude, optionally hydrolysed alkylene oxide addition product then takes place by addition of a stoichiometric amount of one more monobasic inorganic acids, preferably as a dilute aqueous solution. The temperature for hydrolysis and neutralisation can be varied within broad ranges, limits being imposed here by the corrosion resistance of the materials of the neutralisation vessel or the polyol composition. If groups that are susceptible to hydrolysis, such as ester groups for example, are present in the products, neutralisation can be performed at room temperature for example. In such cases it is also advisable to dispense with a preliminary, separate hydrolysis step. Following neutralisation, traces of water introduced by the addition of dilute acids or excess water of hydrolysis can be removed under vacuum. Antioxidants or age resistors can be added to the products during or after neutralisation. No further processing steps, such as for example filtration of the product, take place.

Suitable amino group-containing starter compounds mostly have functionalities (which are understood to be the numbers of Zerewitinoff-active hydrogen atoms present per starter molecule) of 1 to 4. The amino group-containing starter compounds preferably contain at least one primary amino group (—NH₂) and/or secondary amino group and/or tertiary amino group. Their molar masses range from 17 g/mol to approximately 1200 g/mol.

Examples of amino group-containing starter compounds are ammonia, ethanolamine, diethanolamine, triethanolamine, isopropanolamine, diisopropanolamine, ethylenediamine, hexamethylenediamine, aniline, the isomers of toluidine, the isomers of diaminotoluene, the isomers of diaminodiphenylmethane as well as higher-nuclear products produced in the condensation of aniline with formaldehyde to give diaminodiphenylmethane. Mixtures of various amino group-containing starter compounds can of course also be used. Furthermore, mixtures of amino group-containing starters and amino group-free starters can also be used. The content of amino group-containing starters in the starter mixture should be at least 20 mol %. Examples of amino group-free starters are methanol, ethanol, 1-propanol, 2-propanol and higher aliphatic monools, in particular fatty alcohols, phenol, alkyl-substituted phenols, propylene glycol, ethylene glycol, diethylene glycol, dipropylene glycol, 1,2-butanediol, 1,3-butanediol, 1,4-butanediol, hexanediol, pentanediol, 3-methyl-1,5-pentanediol, 1,12-dodecanediol, glycerol, trimethylolpropane, pentaerythritol, sorbitol, sucrose, hydroquinone, catechol, resorcinol, bisphenol F, bisphenol A, 1,3,5-trihydroxybenzene or methylol group-containing condensates of formaldehyde and phenol. In addition, melamine or urea and mannich bases can function as (co)starters.

Pre-prepared alkylene oxide addition products of the cited amino group-containing or amino group-free starter compounds can also be added to the process, in other words polyether polyols having OH values from 20 to 1000 mg KOH/g, preferably 250 to 1000 mg/KOH/g. It is also possible to use polyester polyols having OH values in the range from 6 to 800 mg KOH/g in addition to the starter compounds in the process according to the invention, for the purposes of polyether ester polyol production. Suitable polyester polyols for this purpose can be produced for example from organic dicarboxylic acids having 2 to 12 carbon atoms and polyhydric alcohols, preferably diols, having 2 to 12 carbon atoms, preferably 2 to 6 carbon atoms, by known methods.

Against the background of the scarcity of petrochemical resources and the adverse rating of fossil raw materials in life-cycle assessments, the use of raw materials from sustainable sources is increasingly gaining in importance in the production of suitable polyols for the polyurethane industry too. The method according to the invention opens up a highly cost-effective option for producing such polyols by adding triglycerides, such as for example soya oil, rapeseed oil, palm kernel oil, palm oil, linseed oil, sunflower oil, herring oil, sardine oil, lesquerella oil and castor oil, to the process in amounts from 10 to 80 wt. %, relative to the amount of end product, before or during addition of the alkylene oxides. Polyether ester polyols or polyether ester amide polyols are obtained, into whose structure the oils are completely incorporated, so that they can no longer be detected or detected only in very small amounts in the end product. Surprisingly even oils not containing hydroxyl groups produce homogeneous end products in this variant of the method.

Suitable alkylene oxides are for example ethylene oxide, propylene oxide, 1,2-butylene oxide or 2,3-butylene oxide and styrene oxide. Propylene oxide or a mixture of 100 to 91 wt. % of propylene oxide and 0 to 9 wt. % of ethylene oxide (relative to the amount of epoxides used) is preferably used, with propylene oxide particularly preferably being used exclusively. The various alkylene oxides can be added as a mixture or as blocks. Products having ethylene oxide end blocks are characterised for example by elevated concentrations of primary end groups, which give the systems an elevated isocyanate reactivity, which is desirable for some applications. Pure propylene oxide is most particularly preferably used.

The alkaline crude polyols generally have OH values of 20 to 1000 mg KOH/g, preferably OH values of 28 to 700 mg KOH/g.

The polyols obtainable by the method according to the invention can be used as starting components for the production of solid or foamed polyurethane materials and of polyurethane elastomers. The polyurethane materials and elastomers can also contain isocyanurate, allophanate and biuret structural units. The production of isocyanate prepolymers is likewise possible, in the production of which a molar ratio of isocyanate groups to hydroxyl groups of greater than 1 is used, so that the product contains free isocyanate functionalities. These are only converted on production of the actual end product, in one or more steps.

In order to produce such materials or reaction products, the polyols according to the invention are optionally mixed with further isocyanate-reactive components and reacted with organic polyisocyanates, optionally in the presence of blowing agents in the presence of catalysts, optionally in the presence of other additives such as cell stabilisers for example.

### EXAMPLES

### Raw Materials Used

Irganox® 1076: Octadecyl-3-(3,5-di-tert-butyl-4-hydroxyphenyl)propionate

The OH values were determined in accordance with the instructions in DIN 53240.

The turbidity values were determined in accordance with method 180.1 of the USEPA (United States Environmental Protection Agency). The unit of measurement is NTUs (nephelometric turbidity units).

The viscosity was determined in accordance with DIN ISO 3219.

### Example 1

1046.1 g of ethylene diamine were introduced into a 10-litre laboratory autoclave under a nitrogen atmosphere. After closing the filling pipe, residual oxygen was removed by filling the autoclave three times with nitrogen at 3 bar and then releasing the overpressure down to atmospheric pressure. The contents were heated to 150° C. whilst stirring (450 rpm), and 3711.5 g of propylene oxide were metered into the autoclave over a period of 3 h. The mixture was allowed to react for 1 h and then cooled to 80° C. After adding 2.815 g of a 44.82 wt. % aqueous solution of KOH, the water was removed under vacuum (20 mbar) at 150° C. over a period of 1 h by stripping with nitrogen (50 ml/min). Then 1244.2 g of propylene oxide were introduced over a period of 2.5 h. This was followed by a post-reaction time of 1.5 h. After a curing time of 30 min under vacuum at 15 mbar and cooling to room temperature, four portions of approximately 1300 g each of the resulting reaction mixture were removed from the batch for neutralisation tests (Examples 1A to 1D). The catalyst concentration (KOH) in the resulting reaction mixture was 210 ppm.

### Example 1A

### Comparison

2.028 g of 11.82% sulfuric acid, corresponding to 0.50 mol of sulfuric acid per mol of KOH, were added to 1305.2 g of the resulting reaction mixture from Example 1 at 80° C. and the mixture was stirred for 1 h at 80° C. After adding 0.88 g of Irganox® 1076 the product was dewatered for 1 h at 18 mbar (water jet vacuum) and then for 3 h at 110° C. and 1 mbar. The product had a turbidity of 0.45 NTU.

### Example 1B

### Comparison

4.064 g of 11.82% sulfuric acid, corresponding to 1.00 mol of sulfuric acid per mol of KOH, were added to 1307.6 g of the resulting reaction mixture from Example 1 at 80° C. and the mixture was stirred for 1 h at 80° C. After adding 0.885 g of Irganox® 1076 the product was dewatered for 1 h at 18 mbar (water jet vacuum) and then for 3 h at 110° C. and 1 mbar. The product had a turbidity of 1.54 NTU.

### Example 1C

2.981 g of 10.35% nitric acid, corresponding to 1.00 mol of nitric acid per mol of KOH, were added to 1307.3 g of the resulting reaction mixture from Example 1 at 80° C. and the mixture was stirred for 1 h at 80° C. After adding 0.880 g of Irganox® 1076 the product was dewatered for 1 h at 18 mbar (water jet vacuum) and then for 3 h at 110° C. and 1 mbar. The product had a turbidity of 0.29 NTU.

### Example 1D

2.416 g of 20.35% perchloric acid, corresponding to 1.00 mol of perchloric acid per mol of KOH, were added to 1309.5 g of the resulting reaction mixture from Example 1 at 80° C. and the mixture was stirred for 1 h at 80° C. After adding 0.880 g of Irganox® 1076 the product was dewatered for 1 h at 18 mbar (water jet vacuum) and then for 3 h at 110° C. and 1 mbar. The product had a turbidity of 0.35 NTU.

### Example 2

1049 g of ethylene diamine were introduced into a 10-litre laboratory autoclave under a nitrogen atmosphere. After closing the filling pipe, residual oxygen was removed by filling the autoclave three times with nitrogen at 3 bar and then releasing the overpressure down to atmospheric pressure. The contents were heated to 150° C. whilst stirring (450 rpm), and 3735 g of propylene oxide were metered into the autoclave over a period of 3 h. The mixture was allowed to react for 1 h and then cooled to 80° C. After adding 6.922 g of a 44.82 wt. % aqueous solution of KOH, the water was removed under vacuum (20 mbar) at 150° C. over a period of 1 h by stripping with nitrogen (50 ml/min). Then 1252.2 g of propylene oxide were introduced over a period of 1 h. This was followed by a post-reaction time of 1.5 h. After a curing time of 30 min under vacuum at 15 mbar and cooling to room temperature, four portions of the resulting reaction mixture in amounts from approximately 1180 g to approximately 1450 g were removed from the batch for neutralisation tests (Examples 2A to 2D). The catalyst concentration (KOH) of the resulting reaction mixture was 510 ppm.

### Example 2A

### Comparison

4.509 g of 11.82% sulfuric acid, corresponding to 0.51 mol of sulfuric acid per mol of KOH, were added to 1183.5 g of the resulting reaction mixture from Example 2 at 80° C. and the mixture was stirred for 1 h at 80° C. After adding 0.792 g of Irganox® 1076 the product was dewatered for 1 h at 18 mbar (water jet vacuum) and then for 3 h at 110° C. and 1 mbar. The product had a turbidity of 1.61 NTU.

### Example 2B

### Comparison

8.971 g of 11.82% sulfuric acid, corresponding to 1.00 mol of sulfuric acid per mol of KOH, were added to 1179.3 g of the resulting reaction mixture from Example 2 at 80° C. and the mixture was stirred for 1 h at 80° C. After adding 0.799 g of Irganox® 1076 the product was dewatered for 1 h at 18 mbar (water jet vacuum) and then for 3 h at 110° C. and 1 mbar. The product had a turbidity of 2.39 NTU.

### Example 2C

8.159 g of 10.35% nitric acid, corresponding to 1.00 mol of nitric acid per mol of KOH, were added to 1450.8 g of the resulting reaction mixture from Example 2 at 80° C. and the mixture was stirred for 1 h at 80° C. After adding 0.976 g of Irganox® 1076 the product was dewatered for 1 h at 18 mbar (water jet vacuum) and then for 3 h at 110° C. and 1 mbar. The product had a turbidity of 0.91 NTU.

### Example 2D

6.603 g of 10.35% perchloric acid, corresponding to 1.00 mol of perchloric acid per mol of KOH, were added to 1452.0 g of the resulting reaction mixture from Example 2 at 80° C. and the mixture was stirred for 1 h at 80° C. After adding 0.980 g of Irganox® 1076 the product was dewatered for 1 h at 18 mbar (water jet vacuum) and then for 3 h at 110° C. and 1 mbar. The product had a turbidity of 0.78 NTU.

### Example 3

1025.2 g of ethylene diamine were introduced into a 10-litre laboratory autoclave under a nitrogen atmosphere. After closing the filling pipe, residual oxygen was removed by filling the autoclave three times with nitrogen at 3 bar and then releasing the overpressure down to atmospheric pressure. The contents were heated to 150° C. whilst stirring (450 rpm), and 3725.7 g of propylene oxide were metered into the autoclave over a period of 3 h. The mixture was allowed to react for 1 h and then cooled to 80° C. After adding 13.668 g of a 44.82 wt. % aqueous solution of KOH, the water was removed under vacuum (20 mbar) at 150° C. over a period of 1 h by stripping with nitrogen (50 ml/min). Then 1249.1 g of propylene oxide were introduced over a period of 1 h. This was followed by a post-reaction time of 1.5 h. Then the alkaline crude product was freed from volatile components for a further 30 min at 150° C. under vacuum at 15 mbar. After cooling to room temperature, three portions of approximately 1300 g each of the resulting reaction mixture were removed for neutralisation tests (Examples 3A to 3C). The catalyst concentration (KOH) was 1020 ppm.

### Example 3A

### Comparison

9.969 g of 11.87% sulfuric acid, corresponding to 0.50 mol of sulfuric acid per mol of KOH, were added to 1327.4 g of the resulting reaction mixture from Example 3 at 80° C. and the mixture was stirred for 1 h at 80° C. After adding 0.891 g of Irganox® 1076 the product was dewatered for 1 h at 18 mbar (water jet vacuum) and then for 3 h at 110° C. and 1 mbar. The product had a turbidity of 2.34 NTU.

### Example 3B

### Comparison

19.532 g of 11.87% sulfuric acid, corresponding to 1.00 mol of sulfuric acid per mol of KOH, were added to 1299.8 g of the resulting reaction mixture from Example 3 at 80° C. and the mixture was stirred for 1 h at 80° C. After adding 0.891 g of Irganox® 1076 the product was dewatered for 1 h at 18 mbar (water jet vacuum) and then for 3 h at 110° C. and 1 mbar. The product had a turbidity of 4.30 NTU.

### Example 3C

11.988 g of 20.35% perchloric acid, corresponding to 1.00 mol of perchloric acid per mol of KOH, were added to 1302.3 g of the resulting reaction mixture from Example 3 at 80° C. and the mixture was stirred for 1 h at 80° C. After adding 0.875 g of Irganox® 1076 the product was dewatered for 1 h at 18 mbar (water jet vacuum) and then for 3 h at 110° C. and 1 mbar. The product had a turbidity of 1.01 NTU.

### Example 4

762.4 g of ethylene diamine were introduced into a 10-litre laboratory autoclave under a nitrogen atmosphere. After closing the filling pipe, residual oxygen was removed by filling the autoclave three times with nitrogen at 3 bar and then releasing the overpressure down to atmospheric pressure. The contents were heated to 150° C. whilst stirring (450 rpm), and 2763.8 g of propylene oxide were metered into the autoclave over a period of 3 h. The mixture was allowed to react for 1 h and then cooled to 80° C. After adding 6.758 g of a 44.82 wt. % aqueous solution of KOH, the water was removed under vacuum (20 mbar) at 150° C. over a period of 1 h by stripping with nitrogen (50 ml/min). Then 2473.7 g of propylene oxide were introduced over a period of 2.5 h. This was followed by a post-reaction time of 1.5 h. Then the product was freed from volatile components for a further 30 min at 150° C. under vacuum at 15 mbar. The catalyst concentration (KOH) in the resulting reaction mixture was 506 ppm.

### Example 4A

8.228 g of 20.35% perchloric acid, corresponding to 1 mol of perchloric acid per mol of KOH, were added to 1844.1 g of the resulting reaction mixture from Example 4 at 80° C. and the mixture was stirred for 1 h at 80° C. After adding 1.250 g of Irganox® 1076 the product was dewatered for 1 h at 18 mbar (water jet vacuum) and then for 3 h at 110° C. and 1 mbar. The product had a turbidity of 0.54 NTU.

### Example 5

### Comparison

763.1 g of ethylene diamine were introduced into a 10-litre laboratory autoclave under a nitrogen atmosphere. After closing the filling pipe, residual oxygen was removed by filling the autoclave three times with nitrogen at 3 bar and then releasing the overpressure down to atmospheric pressure. The contents were heated to 150° C. whilst stirring (450 rpm), and 2766.3 g of propylene oxide were metered into the autoclave over a period of 3 h. The mixture was allowed to react for 1 h and then cooled to 80° C. After adding 50.22 g of a 44.82 wt. % aqueous solution of KOH, the water was removed under vacuum (20 mbar) at 150° C. over a period of 1 h by stripping with nitrogen (50 ml/min). Then 2476.0 g of propylene oxide were introduced over a period of 2.0 h. This was followed by a post-reaction time of 1.5 h. After cooling to 80° C., 540 g of water were added, followed by 165.8 g of 11.87% sulfuric acid. After stirring at 80° C. for 0.5 h, 4.02 g of IRGANOX® 1076 were added, the water was removed by distillation, and the residue was freed from volatile components for 3 h at 110° C. under vacuum (1 mbar). After filtration through a depth filter (T 750) at 80° C., a clear product was obtained.

The results of the tests are summarised in Table 1.

TABLE 1

| | Measured OH value | Turbidity |
|---|---|---|
| Example | [mg KOH/g] | [NTU] |
| 1 A (comparison) | 626 | 0.45 |
| 1 B (comparison) | 625 | 1.54 |
| 1 C | 625 | 0.29 |
| 1 D | 625 | 0.35 |
| 2 A (comparison) | 624 | 1.61 |
| 2 B (comparison) | 624 | 2.39 |
| 2 C | 623 | 0.91 |
| 2 D | 624 | 0.78 |
| 3 A (comparison) | 622 | 2.34 |
| 3 B (comparison) | 620 | 4.30 |
| 3 C | 621 | 1.01 |
| 4 A | 470 | 0.54 |
| 5 (comparison) | 471 | n.d. |

n.d. = not determined

Production of MDI Prepolymers from the Polyols Obtained According to Example 4A and 5

Polymeric MDI mixture: The polymeric MDI mixture used in the examples below consisted of 44 wt. % of monomeric diphenylmethane diisocyanate (MDI) and 56 wt. % of polymeric MDI. The monomeric MDI component was made up of 96 wt. % 4,4′-MDI and 4 wt. % 2,4′-MDI.

### Example 6

95.95 parts by weight of a polymeric MDI mixture were reacted with 4.05 parts by weight of the polyether according to Example 4A at 80° C. within 2 h. After cooling to 21° C. a prepolymer was obtained with an NCO content of 27.3 wt. % and a viscosity at 25° C. of 2800 mPas.

Then the prepolymer was stored for 5 days at 21° C. and the viscosity was determined again. The viscosity after storage was 24,360 mPas at 25° C. The viscosity thus increased by a factor of 8.7 over a storage period of 5 days at 21° C.

### Example 7

### Comparison

95.96 parts by weight of a polymeric MDI mixture were reacted with 4.04 parts by weight of the polyether according to comparative example 5 at 80° C. within 2 h. After cooling to 21° C. a prepolymer was obtained with an NCO content of 27.1 wt. % and a viscosity at 25° C. of 4100 mPas.

Then the prepolymer was stored for 5 days at 21° C. and the viscosity was determined again. The viscosity after storage was 164,000 mPas at 25° C. The viscosity thus increased by a factor of 40 over a storage period of 5 days at 21° C.

The polyols according to the invention exhibit markedly lower turbidity values than the polyols of the comparative examples. Furthermore, an isocyanate group-containing prepolymer produced from a polyol according to the invention (Example 6) has a higher storage stability than a corresponding prepolymer (comparative example 7) based on a polyol produced according to comparative example 5.

1-6. (canceled)

7. A process for preparing polyols comprising
(i) adding alkylene oxide mixtures containing alkylene oxides other than ethylene oxide or a maximum of 9 weight % of ethylene oxide to amino group-containing starter compounds in the presence of a catalyst selected from the group consisting of alkali metal hydroxide, alkali metal hydride, alkaline-earth metal hydride, alkali metal carboxylate, alkaline-earth metal carboxylate, alkaline-earth hydroxide and mixtures thereof,
(ii) neutralizing basic catalyst residues by adding a stoichiometric amount of one or more monobasic inorganic acids, and
(iii) leaving salts formed in the resulting polyol.

8. The method of claim 7, wherein 10 to 80 wt.%, relative to the amount of end product, of triglycerides are added before or during addition of the alkylene oxides.

9. The method of claim 7, wherein perchloric acid and/or nitric acid are used as monobasic acids.

10. The method of claim 7, wherein in step (i) propylene oxide or a mixture of 100 to 91 wt. % of propylene oxide and 0 to 9 wt. % of ethylene oxide is added to amino group-containing starter compounds in the presence of a catalyst selected from the group consisting of alkali metal hydroxide, alkali metal hydride, alkaline-earth metal hydride, alkali metal carboxylate, alkaline-earth metal carboxylate, alkaline-earth hydroxide, and mixtures thereof.

11. A polyol prepared by the process of claim 7.

12. A polyurethane prepared from the polyol of claim 11.
